(19) United States
(12) Patent Application Publication
(10) Pub. No.: US 2023/0053162 A1
(43) Pub. Date: Feb. 16, 2023

(54) METHOD FOR ROBOT TO SIMULATE PASSIVE MECHANICAL STATE OF HUMAN LIMB MUSCLES

(71) Applicant: JIANGSU SMART-WALK INTELLIGENCE TECHNOLOGYCO.,LTD., Nanjing (CN)

(72) Inventors: Keshu Cai, Nanjing (CN); Xiaorong Guan, Nanjing (CN); Meng Zhu, Nanjing (CN); Ji Cui, Nanjing (CN); Zhong Li, Nanjing (CN)

(73) Assignee: JIANGSU SMART-WALK INTELLIGENCE TECHNOLOGY CO., LTD., Nanjing (CN)

(21) Appl. No.: 17/792,394

(22) PCT Filed: Jun. 11, 2020
(86) PCT No.: PCT/CN2020/095523

§ 371 Date: Jul. 12, 2022

(30) Foreign Application Priority Data
May 21, 2020 (CN) 202010435950.2

(51) Int. Cl. B25J 9/02; A61F 5/01

(52) U.S. Cl. CPC B25J 9/02; A61F 5/013

(57) ABSTRACT

A method for a robot to simulate the passive mechanical state of human limb muscles, comprising a method for simulating different degrees of tensile force in bending the elbow or bending the knee in the human body, and a method for simulating different degrees of tensile force in extending the elbow or extending the knee in the human body. The robot is provided with, sequentially connected, a base (1), a shoulder joint assembly, an upper arm (5), an elbow joint assembly, a forearm (14), and a palm (16). The shoulder joint assembly is able to drive the upper arm (5) to rotate in all directions, and the elbow joint assembly is able to drive the forearm (14) to bend or extend.

### BACKGROUND OF THE INVENTION

### 1. Technical Field

The invention relates to the technical field of robot simulation, in particular to a method for simulating the passive mechanics state of muscles of human limbs with a robot.

### 2. Description of Related Art

The passive mechanics state of the muscles of human limbs is mainly manifested in the tone state exhibited when the muscles of the upper or lower limbs of the human body are passively stretched. In medical, kinematic, and other related fields, the muscle strength is generally graded. With the modified Ashworth scale commonly used in clinical medicine as the muscle tone grading standard, the muscle tone of human limbs may be classified into six grades, which are respectively grade 0, grade 1, grade 1+, grade 2, grade 3 and grade 4.

When the nerves, especially central nerves, of the human body are injured, the nerve control system will be impaired, muscles dominated by the neural control system will show different degrees of tone, and different degrees of resistance will be generated when the muscles are stretched. For the sake of an accurate diagnosis and a reasonable treatment, body science professionals need to check, test and comprehensively analyze the tone shown by the muscles when the muscles are passively stretched.

At present, students majoring in body science such as medicine and kinesiology will not be exposed to patients to start to practically learn a method for checking the tone shown by human muscles when the muscles are passively moved, until they have an internship, so it is relatively late for these students to learn and master this skill; and these students, as well as junior body science professionals, will not master this method until they have examined a large number of patients in clinical practice, so the learning progress, learning effect and learning systematicness are seriously compromised.

### BRIEF SUMMARY OF THE INVENTION

The objective of the invention is to provide a method for simulating the passive mechanics state of muscles of human limbs with a robot, which provides a standard learning and practice platform for students majoring in medicine and kinesiology and junior body science professionals to master basic clinical physical examination skills and analyze medical and kinematic problems and is of great significance for raising teaching standards, perfecting teaching methods, and improving teaching effects.

The technical scheme adopted by the invention is as follows:

a method for simulating the passive mechanics state of muscles of human limbs with a robot, comprising a method for simulating different grades of muscle tone of human elbow or knee flexion with the robot and a method for simulating different grades of muscle tone of human elbow or knee extension with the robot, wherein the robot comprises a base, a shoulder joint assembly, a big arm, an elbow joint assembly, a small arm and a palm; the shoulder joint assembly comprises a shoulder joint fixed part and a shoulder joint moving part, the shoulder joint fixed part has an end fixed on the base and an end connected to the shoulder joint moving part, and the shoulder joint moving part is fixedly connected to the big arm and is able to drive the big arm to rotate in all directions; the elbow joint assembly comprises a drive motor and a motor reducer, an input end of the motor reducer is connected to the drive motor, the big arm and the small arm are located on two sides of an output shaft end of the motor reducer respectively, the motor reducer is fixed on the big arm and drives the small arm to rotate around a motor shaft, the palm is fixedly connected to a tail end of the small arm, and the big arm, the small arm and the palm are located on a same axis;

pressure sensors are disposed on a palm side and a back side of a front end of the small arm of the robot and are used for measuring the magnitude of a force applied to the front end of the small arm by an operator; the shoulder joint assembly is provided with a gyro sensor used for detecting a rotation angle of a shoulder joint; the elbow joint assembly is provided with an angle sensor used for detecting a rotation angle of the small arm; and the pressure sensors, the gyro sensor and the angle sensor are connected to a controller unit, the drive motor is connected to the controller unit through a motor driver, and the pressure sensors, the gyro sensor, the angle sensor, the drive motor, the motor driver and the controller unit form a control system.

Furthermore, wherein the method for simulating different grades of muscle tone of human elbow flexion comprises:

grade 0: placing, by the operator, the robot in a position where the shoulder joint and an elbow joint hang down naturally and the palm faces forward, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately; and smoothly completing an extension motion of the elbow joint within a full range of 145°-0° by the operator without an obvious resistance;

grade 1: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 10°; and when the elbow joint is moved to an angle less than 10°, detecting, by the controller unit, angle information fed back by the angle sensor to control the drive motor to increase a moment of rotation, so that the operator feels a 0N-5N resistance when moving the elbow joint from 10° (not included) to 0°;

grade 1+: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 60°; and when the elbow joint is moved to an angle less than 60°, detecting, by the controller unit, angle information fed back by the angle sensor to control the drive motor to increase the moment of rotation, so that the operator feels a resistance greater than 5N and less than 10N when moving the elbow joint from 60° (not included) to 0°;

grade 2: placing, by the operator, the robot in a position where the shoulder joint and the elbow joint hang down naturally and the pawl faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 120°; and when the elbow joint is moved to an angle less than 120°, detecting, by the controller unit, angle information fed back by the angle sensor to control the drive motor to increase the moment of rotation, so that the operator feels a 10N-30N resistance when moving the elbow joint from 120° (not included) to 0°;

grade 3: placing, by the operator, the robot in a position where the shoulder joint and the elbow joint hang down naturally and the palm faces forward, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; and next, quickly extending the elbow joint by the operator by moving both hands coordinately, wherein the operator feels a resistance greater than 30N and less than 80N when moving the elbow joint within the full range of 145°-0°;

grade 4: placing, by the operator, the robot in a position where the shoulder joint and the elbow joint hang down naturally and the palm face forwards, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator feels a 80N-200N resistance when moving the elbow joint from 145° to 110°; and when the elbow joint is moved to an angle less than 110°, detecting, by the controller unit, angle information fed back by the angle sensor to control the drive motor to increase the moment of rotation, so that the elbow joint is locked and will not be extended by the operator;

the method for simulating different grades of muscle tone of human elbow flexion is also suitable for simulating different grades of muscle tone of human knee flexion; and when the method is used for simulating different grades of muscle tone of human knee flexion, the shoulder joint is equivalent to a hip joint, and the elbow joint is equivalent to a knee joint.

Furthermore, wherein the method for simulating different grades of muscle tone of human elbow extension with the robot comprises:

grade 0: placing, by the operator, the robot in a position where the shoulder joint and an elbow joint hang down naturally and the palm faces forwards, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when a controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately; and smoothly completing, by the operator, a flexion motion of the elbow joint within a full range of 0°-145° without an obvious resistance;

grade 1: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 135°; and when the elbow joint is moved to an angle greater than 135°, detecting, by the controller unit, angle information of the elbow joint fed back by the angle sensor to control the drive motor to increase a moment of rotation, so that the operator feels a 0N-5N resistance when moving the elbow joint from 135° (not included) to 145°;

Grade 1+: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 85°; and when the elbow joint is moved to an angle greater than 85°, detecting, by the controller unit, angle information fed back by the angle sensor to drive the drive motor to increase the moment of rotation, so that the operator feels a resistance greater than 5N and less than 10N when moving the elbow joint from 85° (not included) to 145°;

grade 2: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 30°; and when the elbow joint is moved to an angle greater than 30°, detecting, by the controller unit, angle information fed back by the angle sensor to control the drive motor to increase the moment of rotation, so that the operator feels a 10N-30N resistance when moving the elbow joint from 30° (not included) to 145°;

grade 3: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; and next, driving the small arm to move in a flexion direction by the operator by moving both hands coordinately, wherein the operator feels a resistance greater than 30N and less than 80N when moving the elbow joint within the full range of 0°-145°;

grade 4: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator feels a 80N-200N resistance when moving the elbow joint from 0° to 60°; and when the elbow joint is moved to an angle greater than 60°, detecting, by the controller unit, angle information fed back by the angle sensor to control the drive motor to increase the moment of rotation, so that the elbow joint is locked and will not be extended by the operator;

the method for simulating different grades of muscle tone of human elbow extension is also suitable for simulating different grades of muscle tone of human knee extension; and when the method is used for simulating different grades of muscle tone of human knee extension, the shoulder joint is equivalent to a hip joint, and the elbow joint is equivalent to a knee joint.

Furthermore, wherein the control system further comprises a human-machine interaction system and the motor driver, and the human-machine interaction system exchanges control and motion information with the controller unit; the motor driver and the drive motor forms a drive unit;

the controller unit determines whether the rotation angle of the shoulder joint of the robot is within a set range according to output information of the gyro sensor, and sends a determining result to the human-machine interaction system to be displayed;

the controller unit determines whether a rotation angle of the elbow joint of the robot is within a set range according to output information of the angle sensor, and sends a determining result to the human-machine interaction system to be displayed;

the controller unit sets a starting torque of the drive motor according to different tone grade control instructions, and sends the starting torque to the motor driver, so that the motor driver drives the drive motor to operate; and during a motion performing process, the moment of rotation of the drive motor is adjusted after rotation angle information of the elbow joint fed back by the angle sensor is received, and then the adjusted moment of rotation is sent to the motor driver, so that the motor driver drives the drive motor to operate;

the human-machine interaction system comprises a display unit, an alarm unit and a plurality of button units, wherein the display unit is used for displaying angles of the shoulder joint and the elbow joint detected by the gyro sensor and the angle sensor; the alarm unit is used for giving an alarm when the controller unit determines that the rotation angle of the elbow joint or the shoulder joint is out of the set range; the button units exchange control and motion information with the controller unit, and comprise an elbow flexion mode button unit, an elbow extension mode button unit, operation button units of different grades in an elbow flexion mode, and operation button units of different grades in an elbow extension mode, and the operator selects different grades of elbow flexion or extension for simulation through the operation button units; the button units further comprise a “start” button unit, a “stop” button unit, an “emergency stop” button unit, an “emergency stop revoke” button unit and a “reset” button unit; the “emergency stop” button unit is used for an emergency stop when the rotation angle of the elbow joint or the shoulder joint of the robot is out of the set range; and the “emergency stop revoke” button unit and the “reset” button unit are used for returning the elbow joint or the shoulder joint of the robot to an initial position after the emergency stop.

Furthermore, wherein the controller unit is a PLC, and the human-machine interaction system is an industrial touch screen.

Furthermore, wherein a control process of the control system comprises the following steps that are performed in sequence:

S1, initialization, namely, login of the operator;

S2, mode section: selecting, by the operator, a passive mechanics mode to be simulated through a mode button;

S3, grade selection: selecting, by the operator, a grade for simulation in the determined mode through a grade button;

S4: condition determination: determining whether the shoulder joint and the elbow joint of the robot are within the set ranges under the selected grade and whether the corresponding pressure sensor detects a pressure; and if the conditions are met, performing the next step;

S5: start: starting the drive motor;

S6: running: performing corresponding motions by the robot;

S7: out-of-limit determination: during the running process, feeding back, by the angle sensor and the gyro sensor, the rotation angle of the shoulder joint and the rotation angle of the elbow joint in real time to determine whether the shoulder joint and the elbow joint are out of the set ranges; if the shoulder joint and the elbow joint are out of the set ranges, giving an alarm by the human-machine interaction system, and sequentially performing, by the operator, an emergency stop, an emergency stop revocation and a reset operation, and returning to S4; or if the shoulder joint and the elbow joint are not out of the set ranges, continuing this step;

S8: end determination: after the controller unit executes a simulation program of the corresponding grade, confirming, by the operator, whether the simulation process is ended; if the simulation process is not ended, returning to S6; or, if the simulation process is ended, performing the next step; and

S9: end: ending the simulation program by the controller unit.

Furthermore, wherein a stop cover is disposed on an outer side of a small arm flange of the robot and is used to limit a rotation angle of the small arm flange within 0°-145°, so that a motion range of an elbow joint is limited.

Furthermore, wherein a shoulder joint moving part of the robot is a universal ball damping joint link having a fixed end connected to a should joint fixed part and a free end fixedly connected to the big arm, and the universal ball damping joint link drives the big arm to rotate in all directions.

Furthermore, wherein the motor reducer of the robot is a harmonic reducer, the drive motor is connected to the harmonic reducer through a reducer flange, a big arm flange is disposed at an end of the big arm, a small arm flange is disposed at an end of the small arm, the big arm flange is fixedly connected to a rigid wheel at an output end of the harmonic reducer, and the small arm flange is disposed on an outer side of the big arm flange and is fixedly connected to a flexible wheel at the output end of the harmonic reducer.

The invention has the following beneficial effects: the method of this application is used for simulating the expressions of different grades of muscle tone that may appear when fatigued muscles are passively moved, and users can observe, feel and repeatedly ponder and learn these expressions of different grades of muscle tone appearing when the fatigued muscles are passively moved, to quickly master the method for examining human muscle tone so as to improve the practical skill; and the application provides a standard learning and practice platform for colleges to carry out visual and targeted practice simulation education for students majoring in medicine and kinesiology, as well as junior medical staff, and is of great significance for raising teaching standards, perfecting teaching methods, and improving teaching effects of the colleges.

### BRIEF DESCRIPTION OF THE SEVERAL VIEWS OF THE DRAWINGS

FIG. 1 is a three-dimensional structural view of a robot according to the

invention.

FIG. 2 is an exploded view of an elbow joint assembly of the robot according to the invention.

FIG. 3 is an exploded view of a shoulder joint assembly of the robot according to the invention.

FIG. 4 is a structural view of a stop cover of the robot according to the

invention.

FIG. 5 is a schematic diagram of the robot in a position where an upper limb hangs down naturally and a palm faces forward according to the invention.

FIG. 6 is a schematic diagram of the robot in a position where a shoulder joint is in 45° of anteflexion, an elbow joint is in 145° of flexion and the palm faces the shoulder joint according to the invention.

FIG. 7 is a schematic diagram of the robot in a position where the shoulder joint in 45° of anteflexion, the elbow joint is extended, and the palm faces forward according to the invention.

FIG. 8 is a structural diagram of a control system of the robot according to the invention.

FIG. 9 is a control flow diagram of the control system of the robot according to the invention.

### DETAILED DESCRIPTION OF THE INVENTION

The technical solutions of the invention will be clearly and completely described below in conjunction with specific embodiments and accompanying drawings. Obviously, the embodiments in the following description are merely illustrative ones, and are not all possible ones of the invention. All other embodiments obtained by those ordinarily skilled in the art according to the following ones without creative labor also fall within the protection scope of the invention.

In the following embodiments, the extension position of the elbow joint is set as a 0° position, and the maximum flexion position of the elbow joint is set as a 145° position. The flexion motion within the full range of 0°-145° refers to a motion of the elbow joint from the extension position, namely the 0° position, to the maximum flexion position, namely the 145° position. The extension motion within the full range of 0°-145° refers to a motion of the elbow joint from the maximum flexion position, namely the 145° position, to the extension position, namely the 0° position.

### Embodiment 1

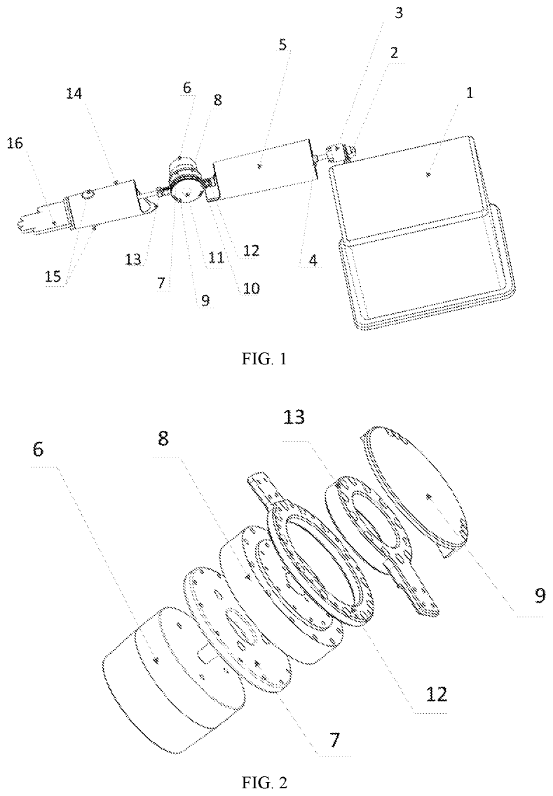

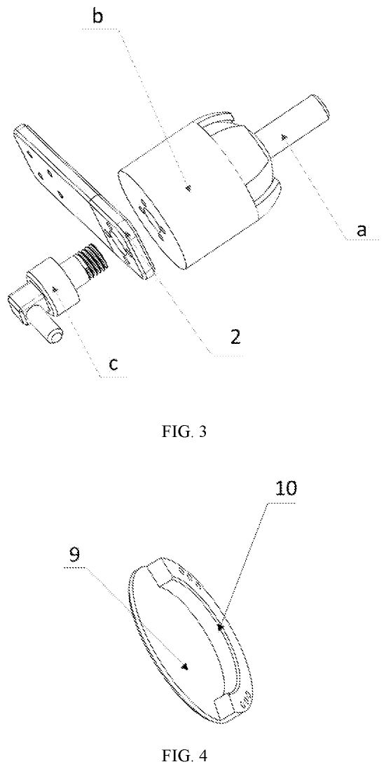

Referring to FIG. 1-FIG. 4, this embodiment provides a robot for simulating the passive mechanics state of muscles of human limbs, comprising a base 1, a shoulder joint assembly, a big arm 5, an elbow joint assembly, a small arm 14 and a palm 16, wherein the shoulder joint assembly comprises a shoulder joint fixed part 2 and a shoulder joint moving part 3, one end of the shoulder joint fixed part 2 is fixed on the base, the other end of the shoulder joint fixed part 2 is connected to the shoulder joint moving part 3, and the shoulder joint moving part 3 is fixedly connected to the big arm 5 and is able to drive the big arm to rotate in all directions; the elbow joint assembly comprises a drive motor 6 and a motor reducer 8, an input end of the motor reducer 8 is connected to the drive motor 6, the big arm 5 and the small arm 14 are located on two sides of an output shaft end of the motor reducer respectively, the motor reducer 8 is fixed on the big arm 5 and drives the small arm 14 to rotate around a motor shaft, the palm 16 is fixedly connected to a tail end of the small arm 14, and the big arm 5, the small arm 14 and the palm 16 are located on the same axis. The structure of the components will be introduced in detail below.

In this embodiment, the base 1 is shaped like a cuboid, and the shoulder joint fixed part 2 is fixed at one corner of an upper end of the cuboid.

In this embodiment, the shoulder joint fixed part 2 is shaped like a rectangular plate, four threaded holes for connecting the shoulder joint fixed part 2 to the base are formed in one end of the plate, and a circular through hole for mounting the shoulder joint moving part 3 is formed in the other end of the plate. The shoulder joint fixed part 2 is fixed on the base 1 with bolts.

In this embodiment, the shoulder joint moving part 3 is a universal ball damping joint link, and preferably, the model of the universal ball damping joint link is 1-150 KGF.CM. The universal ball damping joint link comprises a damping fixed part c, a ball shell b and a ball moving part a, wherein the damping fixed part c is connected to the ball shell b in a threaded manner, the ball shell b is connected to the ball moving part a and is able to rotate with respect to the ball moving part a in all directions, and the big arm 2 is fixed on the ball moving part a and is coaxial with the ball moving part a. A gyro sensor 4 is mounted on the ball moving part a and is used for detecting a rotation angle of the ball moving part a.

In this embodiment, the big arm 5 is shaped like a hollow tube and has two ends being sealed with seal plates, and the seal plate at the end close to an elbow joint is shaped like a concave arc and has a radian matching the elbow joint assembly. The arc-shaped seal plate of the big arm is connected to a big arm flange 12 in an axial direction, and a connecting portion connected to the big arm is disposed on the big arm flange 12 in a radial direction. The small arm 14 is shaped like a hollow tube and has two ends being sealed with seal plates, and the seal plate at the end close to the elbow joint is shaped like a concave arc and has a radian matching the elbow joint assembly. The arc-shaped seal plate of the small arm is connected to a small arm flange in the axial direction, and a connecting portion connected to the big arm is disposed on the small arm flange 13 in the radial direction.

In this embodiment, the motor reducer 8 is preferably a harmonic reducer. The harmonic reducer is directly connected to the drive motor to reduce the installation space, so that the structure of the elbow joint is smaller. The harmonic reducer 8 is connected to the drive motor 6 through a reducer flange 7, and the motor shaft of the drive motor 6 is inserted into a harmonic generator of the harmonic reducer 8. The big arm flange 12 is fixed on a rigid wheel at an output end of the harmonic reducer 8 with screws, the small arm flange 13 is disposed on an outer side of the big arm flange 12, and a rotational clearance is formed between the small arm flange 13 and the big arm flange 12; the small arm flange 13 is fixed on a flexible wheel of the harmonic reducer 8 with screws, and a stop cover 9 is disposed on an outer side of the small arm flange 13 and is fixed on the big arm flange 12 with screws. The reducer flange 7, the big arm flange 12, the small arm flange 13 and the stop cover 9 are coaxially arranged in sequence.

In this embodiment, an arc-shaped stop protrusion 10 is disposed on an outer edge of an inner side of the stop cover 9, provided with a stop cover connecting hole, and fixed on the outer side of the big arm flange 12 with screws, a rotation space for receiving the small arm flange 13 is formed by the big arm flange 12 and the stop cover 9, the small arm drives the small arm flange 13 to rotate in the rotation space, and when the connecting portion of the small arm flange 13 abuts against two ends of the arc-shaped stop protrusion 10, the small arm flange 13 is blocked by the arc-shaped stop protrusion 10 and stops rotating, so that the rotation angle of the small arm 14 is limited to 0°-145°.

In this embodiment, an angle sensor 11 is disposed on one side of the stop cover 9 and is used for detecting a relative rotation angle of the small arm 14 and the big arm 5. Two pressure sensors 15 are mounted on a palm side and a back side of the front end of the small arm 14 respectively and are used for measuring an external force applied by an operator.

As for the robot, if the big arm 5 is replaced with a thigh, the small arm 14 is replaced with a shank and the palm 16 is replaced with a foot sole, the shoulder joint assembly is equivalent to a hip joint assembly, and the elbow joint assembly is equivalent to a knee joint assembly.

The shoulder joint or hip joint of the robot can be manually placed to different positions by the operator. For example, the shoulder joint or hip joint may be in 45° of anteflexion or hang down naturally. By holding the big arm or the ball moving part a, the operator can rotate the ball moving part a in the ball shell b to change the position of the shoulder joint. The drive motor 6 drives the harmonic generator of the harmonic reducer 8 to rotate, and the flexible wheel, as a driven wheel, output the rotation to drive the small arm 14 or the shank to make a motion of one degree of freedom around the elbow joint or knee joint, namely, a flexion and extension motion of the small arm or the shank.

The pressure sensors 15, the gyro sensor 4, the angle sensor 11 and the drive motor 6 are connected to a controller unit, and form a control system together with the controller unit.

### Embodiment 2

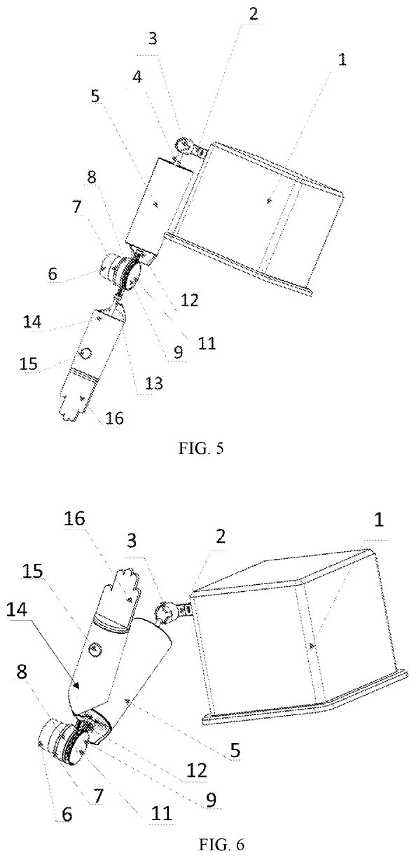

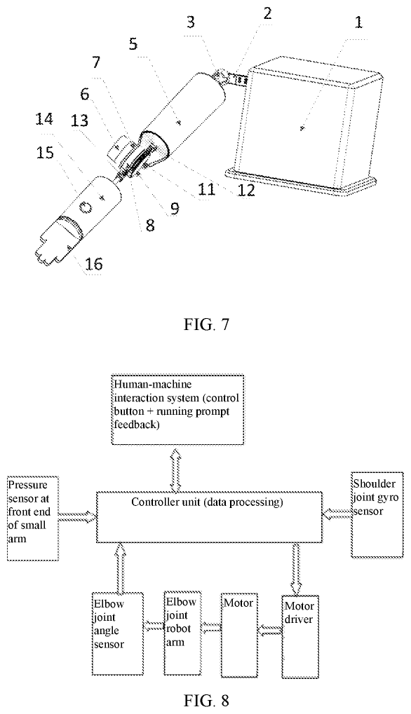

Referring to FIG. 5 and FIG. 6, a method for simulating the passive mechanics state of muscles of human limbs with the robot of the invention will be explained below, with a method for simulating different grades of muscle tone of human elbow flexion with the robot in Embodiment 1 as an example.

The method for simulating different grades of muscle tone of human elbow flexion with the robot of this application comprises:

Grade 0: the operator places the robot in a position where the shoulder joint and the elbow joint hang down naturally and the palm faces forward, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; next, the operator extends the elbow joint quickly by moving both hands coordinately; and the operator completes an extension motion of the elbow joint within the full range of 145°-0° smoothly without feeling an obvious resistance;

Grade 1: the operator places the robot in a position where the shoulder joint and the elbow joint hang down naturally and the palm faces forward, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; next, the operator extends the elbow joint quickly by moving both hands coordinately, wherein the operator will not feel an obvious resistance when moving the elbow joint from 145° to 10°; and when the elbow joint is moved to an angle less than 10°, the controller unit detects angle information fed back by the angle sensor to control the drive motor to increase the moment of rotation, so that the operator feels a 4N resistance when moving the elbow joint from 10° (not included) to 0°;

Grade 1+: the operator places the robot in a position where the shoulder joint and the elbow joint hang down naturally and the palm faces forward, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; next, the operator extends the elbow joint quickly by moving both hands coordinately; the operator will not feel an obvious resistance when moving the elbow joint from 145° to 60°; and when the elbow joint is moved to an angle less than 60°, the controller unit detects angle information fed back by the angle sensor to control the drive motor to increase the moment of rotation, so that the operator feels a 8N resistance when moving the elbow joint from 60° (not included) to 0°;

Grade 2: the operator places the robot in a position where the shoulder joint and the elbow joint hang down naturally and the pawl faces forward, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; next, the operator extends the elbow joint quickly by moving both hands coordinately, wherein the operator will not feel an obvious resistance when moving the elbow joint from 145° to 120°; and when the elbow joint is moved to an angle less than 120°, the controller unit detects angle information fed back by the angle sensor to control the drive motor to increase the moment of rotation, so that the operator feels a 20N resistance when moving the elbow joint from 120° (not included) to 0°;

Grade 3: the operator places the robot in a position where the shoulder joint and the elbow joint hang down naturally and the palm faces forward, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; and next, the operator extends the elbow joint quickly by moving both hands coordinately, wherein the operator feels a resistance over 50N when moving the elbow joint within the full range of 145°-0°;

Grade 4: the operator places the robot in a position where the shoulder joint and the elbow joint hang down naturally and the palm face forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is in 145° of flexion; when the controller unit detects angle information of the shoulder joint and the elbow joint fed back by the gyro sensor and the angle sensor, as well as pressure information fed back by the pressure sensor on the palm side, the operator starts the drive motor; next, the operator extends the elbow joint quickly by moving both hands coordinately, wherein the operator feels a 100N resistance when moving the elbow joint from 145° to 110°; and when the elbow joint is moved to an angle less than 110°, the controller unit detects angle information fed back by the angle sensor to control the drive motor to increase the moment of rotation, so that the elbow joint is locked and will not be extended by the operator.

The method for simulating different grades of muscle tone of elbow flexion is also suitable for simulating different grades of muscle tone of knee flexion; and when the method is used for simulating different grades of muscle tone of knee flexion, the shoulder joint is equivalent to the hip joint, and the elbow joint is equivalent to the knee joint.

### Embodiment 3

The method for simulating the passive mechanics state of muscles of human limbs with the robot of the invention will be explained below, with a method for simulating different grades of muscle tone of human elbow extension with the robot in Embodiment 1 as an example.

The method for simulating different grades of muscle tone of human elbow extension with the robot of this application comprises:

Grade 0: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; next, the operator drives the small arm to move in a flexion direction by moving both hands coordinately; and the operator completes a flexion motion of the elbow joint within the full range of 0°-145° smoothly without feeling an obvious resistance;

Grade 1: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; next, the operator drives the small arm to move in a flexion direction by moving both hands coordinately, wherein the operator will not feel an obvious resistance when moving the elbow joint from 0° to 135°; and when the elbow joint is moved to an angle greater than 135°, the controller unit detects angle information of the elbow joint fed back by the angle sensor to control the drive motor to increase the moment of rotation, so that the operator feels a 4N resistance when moving the elbow joint from 135° (not included) to 145°;

Grade 1+: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; next, the operator drives the small arm to move in a flexion direction by moving both hands coordinately, wherein the operator will not feel an obvious resistance when moving the elbow joint from 0° to 85°; and when the elbow joint is moved to an angle greater than 85°, the controller unit detects angle information fed back by the angle sensor to drive the drive motor to increase the moment of rotation, so that the operator feels a 8N resistance when moving the elbow joint from 85° (not included) to 145°;

Grade 2: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; next, the operator drives the small arm to move in a flexion direction by moving both hands coordinately, wherein the operator will not feel an obvious resistance when moving the elbow joint from 0° to 30°; and when the elbow joint is moved to an angle greater than 30°, the controller unit detects angle information fed back by the angle sensor to control the drive motor to increase the moment of rotation, so that the operator feels a 20N resistance when moving the elbow joint from 30° (not included) to 145°;

Grade 3: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; and next, the operator drives the small arm to move in a flexion direction by moving both hands coordinately, wherein the operator feels a 50N resistance when moving the elbow joint within the full range of 0°-145°;

Grade 4: the operator places the robot in a position where the shoulder joint and the elbow joint hang down naturally and the palm faces forwards, and starts the control system; then, the operator gently lifts the upper limb by holding the back side of the lower end of the big arm of the robot with one hand and holding the front end of the small arm of the robot with the other hand, to make the robot in a position where the shoulder joint is in 45° of anteflexion, 30° of abduction and 30° of intorsion and the elbow joint is extended; when the controller unit detects angle information of the shoulder joint and the elbow joint fed back by the gyro sensor and the angle sensor, as well as pressure information fed back by the pressure sensor on the back side, the operator starts the drive motor; next, the operator drives the small arm to move in a flexion direction by moving both hands coordinately, wherein the operator feels a 100N resistance when moving the elbow joint from 0° to 60°; and when the elbow joint is moved to an angle greater than 60°, the controller unit detects angle information fed back by the angle sensor to control the drive motor to increase the moment of rotation, so that the elbow joint is locked and will not be extended by the operator.

The method for simulating different grades of muscle tone of elbow extension is also suitable for simulating different grades of muscle tone of knee extension; and when the method is used for simulating different grades of muscle tone of knee extension, the shoulder joint is equivalent to the hip joint, and the elbow joint is equivalent to the knee joint.

### Embodiment 4

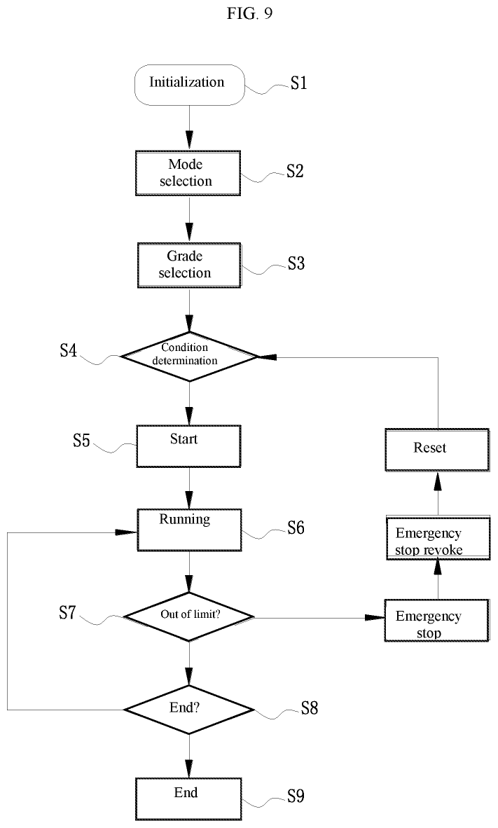

Referring to FIG. 8 and FIG. 9, this embodiment provides a control system and method, which are applied to the method for simulating the passive mechanics state of muscles of human limbs with the robot in Embodiment 2 and Embodiment 3.

The control system is mainly composed of a drive unit, a controller unit, a sensor unit and a human-machine interaction system. The drive unit comprises a drive motor 6 and a motor driver. The sensor unit comprises an angle sensor 11, a pressure sensor 15 and a gyro sensor 4. In this embodiment, the controller unit is preferably a PLC. In other embodiments, the controller unit may be an MCU system, or a PC-based motion control board. In this embodiment, the human-machine interaction system is preferably an industrial touch screen. In other embodiments, the human-machine interaction system may be other touch screens or a mobile phone APP. The PLC exchanges information with the motor driver of the drive motor 6 based on a communication protocol, the drive motor 6 is controlled by an electric current loop, and the PLC sends control information according to a selected function key on the touch screen. The PLC exchanges information with the industrial touch screen based on a communication protocol.

The gyro sensor 4 sends a detected angle of the shoulder joint to the PLC, the PLC determines whether the shoulder joint of the robot is in a set position according to output information of the gyro sensor 4, and sends a determining result to the human-machine interaction system to be displayed on the industrial touch screen, and an alarm is given when the shoulder joint is not in the set position.

The PLC determines whether the elbow joint of the robot is in a set position according to output information of the angle sensor 11, and sends a determining result to the human-machine interaction system to be displayed on the industrial touch screen, and an alarm is given when the elbow joint is not in the set position.

The PLC sets a starting torque of the drive motor 6 according to different tone grade control instructions, and sends the starting torque to the motor driver, so that the motor driver drives the drive motor 6 to operate; and during the motion performing process, the moment of rotation of the drive motor 6 is adjusted after force information fed back by the pressure sensor 15 and angle information fed back by the angle sensor 11 are received, and then the adjusted moment of rotation is sent to the motor driver, so that the motor driver drives the drive motor 6 to operate.

An “elbow flexion mode” control button and an “elbow extension mode” control button are disposed on the industrial touch screen, and correspond to an “elbow flexion mode” button unit and an “elbow extension mode” button unit respectively; and a “grade 0” control button, a “grade 1” control button, a “grade 1+” control button, a “grade 2” control button, a “grade 3” control button and a “grade 4” control button respectively set in the “elbow flexion mode” and the “elbow extension mode”, and correspond to operation button units of different grades respectively. A “start” control button, a “stop” control button, a “reset” control button, an “emergency stop” control button and an “emergency stop revoke” control button are also disposed on the industrial touch screen and correspond to a “start” button unit, a “stop” button unit, a “reset” button unit, an “emergency stop” button unit and an “emergency stop revoke” button unit respectively. The button units exchange control and motion information with the controller unit.

“Start” means login of an operator. “Stop” means that the controller unit has executed a simulation program of a corresponding grade and the operator confirms that this simulation process has finished. “Reset” means that the elbow joint returns to the initial state from the current state or in an emergency stop. “Emergency stop” means that the operator can press this button to stop the simulation system at any time in the simulation process, particularly in a case where a danger may happen if the device continues to operate. “Emergency stop revoke” means that, in order the guarantee the safety of the device in an alarm or in an emergency stop, a mandatory protection measure is taken to protect the device against damage caused by a further operation by the operator after a fault or a trouble is removed.

The control system in this embodiment is used to control the robot, provided by the invention, to simulate different grades of muscle tone of human elbow extension according to the following process.

The control process of the control system comprises the following steps that are performed in sequence:

S1, initialization, namely, login of the operator: during a specific simulation operation, the operator places the robot in a position where the shoulder joint and the elbow joint hang down naturally and the palm faces forward, and then clicks a “start button” to log in the control system;

S2, mode selection: the operator selects a passive mechanics mode to be simulated through the corresponding mode button, wherein during a specific simulation operation, the operator selects the passive mechanics mode to be simulated by clicking the “elbow flexion mode” control button or the “elbow extension mode” control button;

S3, grade selection: the operator selects the grade for simulation in the determined mode through the corresponding grade button, wherein during a specific simulation operation, the operator selects different grades in the corresponding mode by clicking the “grade 0” control button, the “grade 1” control button, the “grade 1+” control button, the “grade 2” control button, the “grade 3” control button and the “grade 4”;

S4, condition determination: whether the shoulder joint and the elbow joint of the robot are within set ranges under the selected grade and whether the pressure sensor detects a pressure are determined, and if the conditions are not met, S4 is performed; or if all the conditions are met, the next step is performed, wherein during a specific simulation operation, in the elbow extension mode, the set ranges are that the shoulder joint is in 0°-75° of anteflexion and 0°-60° of abduction or 0°-20° of adduction and the elbow joint is extended; and in the elbow flexion mode, the set ranges are that shoulder joint is in 0°-75° of anteflexion and 0°-60° of abduction or 0°-20° of adduction and the elbow joint is in 145° of flexion;

S5, start: the drive motor is started, wherein during a specific simulation operation, the controller unit sets a starting torque of the motor according to different tone grade control instructions, and sends the starting torque to the motor driver, so that the motor driver drives the drive motor to operate;

S6, running: the robot performs corresponding motions, wherein during a specific simulation operation, the robot performs an elbow flexion motion or an elbow extension motion of the selected grade in the selected mode;

S7, out-of-limit determination: during the running process, the rotation angle of the shoulder joint and the rotation angle of the elbow joint are fed back in real time by the angle sensor and the gyro sensor to determine whether the shoulder joint and the elbow joint are out of the set ranges; if the shoulder joint and the elbow joint are out of set ranges, the human-machine interaction system gives an alarm, and the operator sequentially performs an emergency stop, an emergency stop revocation and a reset operation, and S4 is performed; if the shoulder joint and the elbow joint are not out of set ranges, S7 is performed to continue the simulation program, wherein the shoulder joint is in 0°-75° of anteflexion and 0°-60° of abduction or 0°-20° of adduction and the elbow joint is in 0°-145° of flexion;

S8, end determination: after the controller unit executes the simulation program of the corresponding grade, the operator confirms whether the simulation process is ended; if the simulation process is not ended, S6 is performed; or, if the simulation process is ended, the next step is performed; and

S9: end: the controller unit ends the simulation process, and the robot stops working.

By adoption of the control system and method, the operator can easily operate and control the robot to simulate the passive mechanics state of muscles of human limbs, and can clearly feel the magnitude of external force applied to an examinee during an elbow flexion and extension examination, and the practical skill of the operator is improved.

The above embodiments are merely preferred ones of the invention, and are not used to limit the invention. Any modifications, equivalent substitutions and improvements made based on the spirit and principle of the invention should fall within the protection scope of the invention.

What is claimed is:

1. A method for simulating the passive mechanics state of muscles of human limbs with a robot, comprising a method for simulating different grades of muscle tone of human elbow or knee flexion with the robot and a method for simulating different grades of muscle tone of human elbow or knee extension with the robot, wherein the robot comprises a base, a shoulder joint assembly, a big arm, an elbow joint assembly, a small arm and a palm; the shoulder joint assembly comprises a shoulder joint fixed part and a shoulder joint moving part, the shoulder joint fixed part has an end fixed on the base and an end connected to the shoulder joint moving part, and the shoulder joint moving part is fixedly connected to the big arm and is able to drive the big arm to rotate in all directions; the elbow joint assembly comprises a drive motor and a motor reducer, an input end of the motor reducer is connected to the drive motor, the big arm and the small arm are located on two sides of an output shaft end of the motor reducer respectively, the motor reducer is fixed on the big arm and drives the small arm to rotate around a motor shaft, the palm is fixedly connected to a tail end of the small arm, and the big arm, the small arm and the palm are located on a same axis;
pressure sensors are disposed on a palm side and a back side of a front end of the small arm of the robot and are used for measuring the magnitude of a force applied to the front end of the small arm by an operator; the shoulder joint assembly is provided with a gyro sensor used for detecting a rotation angle of a shoulder joint; the elbow joint assembly is provided with an angle sensor used for detecting a rotation angle of the small arm; and the pressure sensors, the gyro sensor and the angle sensor are connected to a controller unit, the drive motor is connected to the controller unit through a motor driver, and the pressure sensors, the gyro sensor, the angle sensor, the drive motor, the motor driver and the controller unit form a control system.

2. The method for simulating the passive mechanics state of muscles of human limbs with a robot according to claim 1, wherein the method for simulating different grades of muscle tone of human elbow flexion comprises:
grade 0: placing, by the operator, the robot in a position where the shoulder joint and an elbow joint hang down naturally and the palm faces forward, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately; and smoothly completing an extension motion of the elbow joint within a full range of 145°-0° by the operator without an obvious resistance;
grade 1: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 10°; and when the elbow joint is moved to an angle less than 10°, detecting, by the controller unit, angle information fed back by the angle sensor to control the drive motor to increase a moment of rotation, so that the operator feels a 0N-5N resistance when moving the elbow joint from 10° (not included) to 0°;
grade 1+: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 60°; and when the elbow joint is moved to an angle less than 60°, detecting, by the controller unit, angle information fed back by the angle sensor to control the drive motor to increase the moment of rotation, so that the operator feels a resistance greater than 5N and less than 10N when moving the elbow joint from 60° (not included) to 0°;
grade 2: placing, by the operator, the robot in a position where the shoulder joint and the elbow joint hang down naturally and the pawl faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 120°; and when the elbow joint is moved to an angle less than 120°, detecting, by the controller unit, angle information fed back by the angle sensor to control the drive motor to increase the moment of rotation, so that the operator feels a 10N-30N resistance when moving the elbow joint from 120° (not included) to 0°;
grade 3: placing, by the operator, the robot in a position where the shoulder joint and the elbow joint hang down naturally and the palm faces forward, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; and next, quickly extending the elbow joint by the operator by moving both hands coordinately, wherein the operator feels a resistance greater than 30N and less than 80N when moving the elbow joint within the full range of 145°-0°;
grade 4: placing, by the operator, the robot in a position where the shoulder joint and the elbow joint hang down naturally and the palm face forwards, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator feels a 80N-200N resistance when moving the elbow joint from 145° to 110°; and when the elbow joint is moved to an angle less than 110°, detecting, by the controller unit, angle information fed back by the angle sensor to control the drive motor to increase the moment of rotation, so that the elbow joint is locked and will not be extended by the operator;
the method for simulating different grades of muscle tone of human elbow flexion is also suitable for simulating different grades of muscle tone of human knee flexion; and when the method is used for simulating different grades of muscle tone of human knee flexion, the shoulder joint is equivalent to a hip joint, and the elbow joint is equivalent to a knee joint.

3. The method for simulating the passive mechanics state of muscles of human limbs with a robot according to claim 1, wherein the method for simulating different grades of muscle tone of human elbow extension with the robot comprises:
grade 0: placing, by the operator, the robot in a position where the shoulder joint and an elbow joint hang down naturally and the palm faces forwards, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when a controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately; and smoothly completing, by the operator, a flexion motion of the elbow joint within a full range of 0°-145° without an obvious resistance;
grade 1: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 135°; and when the elbow joint is moved to an angle greater than 135°, detecting, by the controller unit, angle information of the elbow joint fed back by the angle sensor to control the drive motor to increase a moment of rotation, so that the operator feels a 0N-5N resistance when moving the elbow joint from 135° (not included) to 145°;
Grade 1+: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 85°; and when the elbow joint is moved to an angle greater than 85°, detecting, by the controller unit, angle information fed back by the angle sensor to drive the drive motor to increase the moment of rotation, so that the operator feels a resistance greater than 5N and less than 10N when moving the elbow joint from 85° (not included) to 145°;
grade 2: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 30°; and when the elbow joint is moved to an angle greater than 30°, detecting, by the controller unit, angle information fed back by the angle sensor to control the drive motor to increase the moment of rotation, so that the operator feels a 10N-30N resistance when moving the elbow joint from 30° (not included) to 145°;
grade 3: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; and next, driving the small arm to move in a flexion direction by the operator by moving both hands coordinately, wherein the operator feels a resistance greater than 30N and less than 80N when moving the elbow joint within the full range of 0°-145°;
grade 4: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator feels a 80N-200N resistance when moving the elbow joint from 0° to 60°; and when the elbow joint is moved to an angle greater than 60°, detecting, by the controller unit, angle information fed back by the angle sensor to control the drive motor to increase the moment of rotation, so that the elbow joint is locked and will not be extended by the operator;
the method for simulating different grades of muscle tone of human elbow extension is also suitable for simulating different grades of muscle tone of human knee extension; and when the method is used for simulating different grades of muscle tone of human knee extension, the shoulder joint is equivalent to a hip joint, and the elbow joint is equivalent to a knee joint.

4. The method for simulating the passive mechanics state of muscles of human limbs with a robot according to claim 2 or 3, wherein the control system further comprises a human-machine interaction system and the motor driver, and the human-machine interaction system exchanges control and motion information with the controller unit; the motor driver and the drive motor forms a drive unit;
the controller unit determines whether the rotation angle of the shoulder joint of the robot is within a set range according to output information of the gyro sensor, and sends a determining result to the human-machine interaction system to be displayed;
the controller unit determines whether a rotation angle of the elbow joint of the robot is within a set range according to output information of the angle sensor, and sends a determining result to the human-machine interaction system to be displayed;
the controller unit sets a starting torque of the drive motor according to different tone grade control instructions, and sends the starting torque to the motor driver, so that the motor driver drives the drive motor to operate; and during a motion performing process, the moment of rotation of the drive motor is adjusted after rotation angle information of the elbow joint fed back by the angle sensor is received, and then the adjusted moment of rotation is sent to the motor driver, so that the motor driver drives the drive motor to operate;
the human-machine interaction system comprises a display unit, an alarm unit and a plurality of button units, wherein the display unit is used for displaying angles of the shoulder joint and the elbow joint detected by the gyro sensor and the angle sensor; the alarm unit is used for giving an alarm when the controller unit determines that the rotation angle of the elbow joint or the shoulder joint is out of the set range; the button units exchange control and motion information with the controller unit, and comprise an elbow flexion mode button unit, an elbow extension mode button unit, operation button units of different grades in an elbow flexion mode, and operation button units of different grades in an elbow extension mode, and the operator selects different grades of elbow flexion or extension for simulation through the operation button units; the button units further comprise a “start” button unit, a “stop” button unit, an “emergency stop” button unit, an “emergency stop revoke” button unit and a “reset” button unit; the “emergency stop” button unit is used for an emergency stop when the rotation angle of the elbow joint or the shoulder joint of the robot is out of the set range; and the “emergency stop revoke” button unit and the “reset” button unit are used for returning the elbow joint or the shoulder joint of the robot to an initial position after the emergency stop.

5. The method for simulating the passive mechanics state of muscles of human limbs with a robot according to claim 4, wherein the controller unit is a PLC, and the human-machine interaction system is an industrial touch screen.

6. The method for simulating the passive mechanics state of muscles of human limbs with a robot according to claim 4, wherein a control process of the control system comprises the following steps that are performed in sequence:
S1, initialization, namely, login of the operator;
S2, mode section: selecting, by the operator, a passive mechanics mode to be simulated through a mode button;
S3, grade selection: selecting, by the operator, a grade for simulation in the determined mode through a grade button;
S4: condition determination: determining whether the shoulder joint and the elbow joint of the robot are within the set ranges under the selected grade and whether the corresponding pressure sensor detects a pressure; and if the conditions are met, performing the next step;
S5: start: starting the drive motor;
S6: running: performing corresponding motions by the robot;
S7: out-of-limit determination: during the running process, feeding back, by the angle sensor and the gyro sensor, the rotation angle of the shoulder joint and the rotation angle of the elbow joint in real time to determine whether the shoulder joint and the elbow joint are out of the set ranges; if the shoulder joint and the elbow joint are out of the set ranges, giving an alarm by the human-machine interaction system, and sequentially performing, by the operator, an emergency stop, an emergency stop revocation and a reset operation, and returning to S4; or if the shoulder joint and the elbow joint are not out of the set ranges, continuing this step;
S8: end determination: after the controller unit executes a simulation program of the corresponding grade, confirming, by the operator, whether the simulation process is ended; if the simulation process is not ended, returning to S6; or, if the simulation process is ended, performing the next step; and
S9: end: ending the simulation program by the controller unit.

7. The method for simulating the passive mechanics state of muscles of human limbs with a robot according to claim 1, wherein a stop cover is disposed on an outer side of a small arm flange of the robot and is used to limit a rotation angle of the small arm flange within 0°-145°, so that a motion range of an elbow joint is limited.

8. The method for simulating the passive mechanics state of muscles of human limbs with a robot according to claim 1, wherein a shoulder joint moving part of the robot is a universal ball damping joint link having a fixed end connected to a should joint fixed part and a free end fixedly connected to the big arm, and the universal ball damping joint link drives the big arm to rotate in all directions.

9. The method for simulating the passive mechanics state of muscles of human limbs with a robot according to claim 1, wherein the motor reducer of the robot is a harmonic reducer, the drive motor is connected to the harmonic reducer through a reducer flange, a big arm flange is disposed at an end of the big arm, a small arm flange is disposed at an end of the small arm, the big arm flange is fixedly connected to a rigid wheel at an output end of the harmonic reducer, and the small arm flange is disposed on an outer side of the big arm flange and is fixedly connected to a flexible wheel at the output end of the harmonic reducer.

1. A method for simulating a passive mechanics state of muscles of human limbs with a robot, comprising:
a method for simulating different grades of muscle tone of human elbow or knee flexion with the robot and a method for simulating different grades of muscle tone of human elbow or knee extension with the robot, wherein
the robot comprises a base, a shoulder joint assembly, a big arm, an elbow joint assembly, a small arm and a palm;
the shoulder joint assembly comprises a shoulder joint fixed part and a shoulder joint moving part, the shoulder joint fixed part has an end fixed on the base and an end connected to the shoulder joint moving part, and the shoulder joint moving part is fixedly connected to the big arm and is able to drive the big arm to rotate in all directions;
the elbow joint assembly comprises a drive motor and a motor reducer, an input end of the motor reducer is connected to the drive motor, the big arm and the small arm are located on two sides of an output shaft end of the motor reducer respectively, the motor reducer is fixed on the big arm and drives the small arm to rotate around a motor shaft, the palm is fixedly connected to a tail end of the small arm, and the big arm, the small arm and the palm are located on a same axis;
pressure sensors are disposed on a palm side and a back side of a front end of the small arm of the robot and are used for measuring the magnitude of a force applied to the front end of the small arm by an operator;
the shoulder joint assembly is provided with a gyro sensor used for detecting a rotation angle of a shoulder joint; the elbow joint assembly is provided with an angle sensor used for detecting a rotation angle of the small arm; and
the pressure sensors, the gyro sensor and the angle sensor are connected to a controller unit, the drive motor is connected to the controller unit through a motor driver, and the pressure sensors, the gyro sensor, the angle sensor, the drive motor, the motor driver and the controller unit form a control system;
wherein the method for simulating different grades of muscle tone of human elbow flexion comprises:
grade 0: placing, by the operator, the robot in a position where the shoulder joint and an elbow joint hang down naturally and the palm faces forward, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately; and smoothly completing an extension motion of the elbow joint within a full range of 145°-0° by the operator without an obvious resistance;
grade 1: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 10°, and when the elbow joint is moved to an angle less than 10°, detecting, by the controller unit, angle information fed back by the angle sensor to control the drive motor to increase a moment of rotation, so that the operator feels a 0N-5N resistance when moving the elbow joint from 10° (not included) to 0°,
grade 1+: placing, by the operator, the robot in a position where the shoulder joint and the elbow joint hang down naturally and the palm faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 60°, and when the elbow joint is moved to an angle less than 60°, detecting, by the controller unit, angle information fed back by the angle sensor to control the drive motor to increase the moment of rotation, so that the operator feels a resistance greater than 5N and less than 10N when moving the elbow joint from 60° (not included) to 0°,
grade 2: placing, by the operator, the robot in a position where the shoulder joint and the elbow joint hang down naturally and the pawl faces forward, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator will not feel an obvious resistance when moving the elbow joint from 145° to 120°; and when the elbow joint is moved to an angle less than 120°, detecting, by the controller unit, angle information fed back by the angle sensor to control the drive motor to increase the moment of rotation, so that the operator feels a 10N-30N resistance when moving the elbow joint from 120° (not included) to 0°,
grade 3: placing, by the operator, the robot in a position where the shoulder joint and the elbow joint hang down naturally and the palm faces forward, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; and next, quickly extending the elbow joint by the operator by moving both hands coordinately, wherein the operator feels a resistance greater than 30N and less than 80N when moving the elbow joint within the full range of 145°-0°,
grade 4: placing, by the operator, the robot in a position where the shoulder joint and the elbow joint hang down naturally and the palm face forwards, and starts the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is in 135°-145° of flexion; when the controller unit detects angle information of the shoulder joint and the elbow joint fed back by the gyro sensor and the angle sensor, as well as pressure information fed back by the pressure sensor on the palm side, starting the drive motor; next, quickly extending the elbow joint by the operator by moving both hands coordinately, wherein the operator feels a 80N-200N resistance when moving the elbow joint from 145° to 110°; and when the elbow joint is moved to an angle less than 110°, detecting, by the controller unit, angle information fed back by the angle sensor to control the drive motor to increase the moment of rotation, so that the elbow joint is locked and will not be extended by the operator;
the method for simulating different grades of muscle tone of human elbow flexion is also suitable for simulating different grades of muscle tone of human knee flexion; and when the method is used for simulating different grades of muscle tone of human knee flexion, the shoulder joint is equivalent to a hip joint, and the elbow joint is equivalent to a knee joint
wherein the method for simulating different grades of muscle tone of human elbow extension with the robot comprises:
grade 0: placing, by the operator, the robot in a position where the shoulder joint and an elbow joint hang down naturally and the palm faces forwards, and starting the control system; then, gently lifting an upper limb by the operator by holding a back side of a lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when a controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately; and smoothly completing, by the operator, a flexion motion of the elbow joint within a full range of 0°-145° without an obvious resistance;
grade 1: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 135°; and when the elbow joint is moved to an angle greater than 135°, detecting, by the controller unit, angle information of the elbow joint fed back by the angle sensor to control the drive motor to increase a moment of rotation, so that the operator feels a 0N-5N resistance when moving the elbow joint from 135° (not included) to 145°;
Grade 1+: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended;
when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 85°, and when the elbow joint is moved to an angle greater than 85°, detecting, by the controller unit, angle information fed back by the angle sensor to drive the drive motor to increase the moment of rotation, so that the operator feels a resistance greater than 5N and less than 10N when moving the elbow joint from 85° (not included) to 145°;
grade 2: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator will not feel an obvious resistance when moving the elbow joint from 0° to 30°, and when the elbow joint is moved to an angle greater than 30°, detecting, by the controller unit, angle information fed back by the angle sensor to control the drive motor to increase the moment of rotation, so that the operator feels a 10N-30N resistance when moving the elbow joint from 30° (not included) to 145°;
grade 3: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended; when the controller unit detects angle information of the shoulder joint and the elbow joint fed back by the gyro sensor and the angle sensor, as well as pressure information fed back by the pressure sensor on the back side, starting the drive motor; and next, driving the small arm to move in a flexion direction by the operator by moving both hands coordinately, wherein the operator feels a resistance greater than 30N and less than 80N when moving the elbow joint within the full range of 0°-145°;
grade 4: placing, by the operator, the robot in a position where the shoulder joint and the elbow joint hang down naturally and the palm faces forwards, and starting the control system; then, gently lifting the upper limb by the operator by holding the back side of the lower end of the big arm of the robot with one hand and holding the front end of the small arm of the robot with the other hand, to make the robot in a position where the shoulder joint is in 0°-75° of anteflexion, 0°-60° of abduction or 0°-20° of adduction and 0°-90° of intorsion and the elbow joint is extended;
when the controller unit detects angle information of the shoulder joint and the elbow joint fed back by the gyro sensor and the angle sensor, as well as pressure information fed back by the pressure sensor on the back side, starting the drive motor; next, driving the small arm to move in a flexion direction by the operator by moving both hands coordinately, wherein the operator feels a 80N-200N resistance when moving the elbow joint from 0° to 60°, and when the elbow joint is moved to an angle greater than 60°, detecting, by the controller unit, angle information fed back by the angle sensor to control the drive motor to increase the moment of rotation, so that the elbow joint is locked and will not be extended by the operator;
the method for simulating different grades of muscle tone of human elbow extension is also suitable for simulating different grades of muscle tone of human knee extension; and when the method is used for simulating different grades of muscle tone of human knee extension, the shoulder joint is equivalent to a hip joint, and the elbow joint is equivalent to a knee joint.

2-3. (canceled)

4. The method for simulating the passive mechanics state of muscles of human limbs with a robot according to claim 1, wherein the control system further comprises a human-machine interaction system and the motor driver, and the human-machine interaction system exchanges control and motion information with the controller unit; the motor driver and the drive motor forms a drive unit;
the controller unit determines whether the rotation angle of the shoulder joint of the robot is within a set range according to output information of the gyro sensor, and sends a determining result to the human-machine interaction system to be displayed;
the controller unit determines whether a rotation angle of the elbow joint of the robot is within a set range according to output information of the angle sensor, and sends a determining result to the human-machine interaction system to be displayed;
the controller unit sets a starting torque of the drive motor according to different tone grade control instructions, and sends the starting torque to the motor driver, so that the motor driver drives the drive motor to operate; and during a motion performing process, the moment of rotation of the drive motor is adjusted after rotation angle information of the elbow joint fed back by the angle sensor is received, and then the adjusted moment of rotation is sent to the motor driver, so that the motor driver drives the drive motor to operate;
the human-machine interaction system comprises a display unit, an alarm unit and a plurality of button units, wherein the display unit is used for displaying angles of the shoulder joint and the elbow joint detected by the gyro sensor and the angle sensor; the alarm unit is used for giving an alarm when the controller unit determines that the rotation angle of the elbow joint or the shoulder joint is out of the set range; the button units exchange control and motion information with the controller unit, and comprise an elbow flexion mode button unit, an elbow extension mode button unit, operation button units of different grades in an elbow flexion mode, and operation button units of different grades in an elbow extension mode, and the operator selects different grades of elbow flexion or extension for simulation through the operation button units; the button units further comprise a “start” button unit, a “stop” button unit, an “emergency stop” button unit, an “emergency stop revoke” button unit and a “reset” button unit; the “emergency stop” button unit is used for an emergency stop when the rotation angle of the elbow joint or the shoulder joint of the robot is out of the set range; and the “emergency stop revoke” button unit and the “reset” button unit are used for returning the elbow joint or the shoulder joint of the robot to an initial position after the emergency stop.

5. The method for simulating the passive mechanics state of muscles of human limbs with a robot according to claim 4, wherein the controller unit is a PLC, and the human-machine interaction system is an industrial touch screen.

6. The method for simulating the passive mechanics state of muscles of human limbs with a robot according to claim 4, wherein a control process of the control system comprises the following steps that are performed in sequence:
S1, initialization, namely, login of the operator;
S2, mode section: selecting, by the operator, a passive mechanics mode to be simulated through a mode button;
S3, grade selection: selecting, by the operator, a grade for simulation in the determined mode through a grade button;
S4: condition determination: determining whether the shoulder joint and the elbow joint of the robot are within the set ranges under the selected grade and whether the corresponding pressure sensor detects a pressure; and if the conditions are met, performing the next step;
S5: start: starting the drive motor;
S6: running: performing corresponding motions by the robot;
S7: out-of-limit determination: during the running process, feeding back, by the angle sensor and the gyro sensor, the rotation angle of the shoulder joint and the rotation angle of the elbow joint in real time to determine whether the shoulder joint and the elbow joint are out of the set ranges; if the shoulder joint and the elbow joint are out of the set ranges, giving an alarm by the human-machine interaction system, and sequentially performing, by the operator, an emergency stop, an emergency stop revocation and a reset operation, and returning to S4; or if the shoulder joint and the elbow joint are not out of the set ranges, continuing this step;
S8: end determination: after the controller unit executes a simulation program of the corresponding grade, confirming, by the operator, whether the simulation process is ended; if the simulation process is not ended, returning to S6; or, if the simulation process is ended, performing the next step; and
S9: end: ending the simulation program by the controller unit.

7. The method for simulating the passive mechanics state of muscles of human limbs with a robot according to claim 1, wherein a stop cover is disposed on an outer side of a small arm flange of the robot and is used to limit a rotation angle of the small arm flange within 0°-145°, so that a motion range of an elbow joint is limited.

8. The method for simulating the passive mechanics state of muscles of human limbs with a robot according to claim 1, wherein a shoulder joint moving part of the robot is a universal ball damping joint link having a fixed end connected to a should joint fixed part and a free end fixedly connected to the big arm, and the universal ball damping joint link drives the big arm to rotate in all directions.

9. The method for simulating the passive mechanics state of muscles of human limbs with a robot according to claim 1, wherein the motor reducer of the robot is a harmonic reducer, the drive motor is connected to the harmonic reducer through a reducer flange, a big arm flange is disposed at an end of the big arm, a small arm flange is disposed at an end of the small arm, the big arm flange is fixedly connected to a rigid wheel at an output end of the harmonic reducer, and the small arm flange is disposed on an outer side of the big arm flange and is fixedly connected to a flexible wheel at the output end of the harmonic reducer.
